(19) United States
(12) Patent Application Publication
(10) Pub. No.: US 2014/0227208 A1
(43) Pub. Date: Aug. 14, 2014

(54) COSMETIC COMPOSITIONS CONTAINING SUBSTITUTED AZOLE AND METHODS FOR ALLEVIATING MECHANICALLY-INDUCED SKIN INFLAMMATION

(71) Applicant: The Procter & Gamble Company, Cincinnati, OH (US)

(72) Inventors: Supriya Punyani BAREJA, New Delhi (IN); Stevan David JONES, Singapore (SG); John Erich OBLONG, Loveland, OH (US); Kevin John MILLS, Goshen, OH (US); John Crist BIERMAN, Cincinnati, OH (US)

(73) Assignee: The Procter & Gamble Company, Cincinnati, OH (US)

(21) Appl. No.: 14/167,203

(22) Filed: Jan. 29, 2014

Related U.S. Application Data
(60) Provisional application No. 61/762,562, filed on Feb. 8, 2013.

(51) Int. Cl. A61K 31/4164; A61K 45/06; A61Q 19/10; A61Q 19/00; A61Q 17/04; A61K 8/49

(52) U.S. Cl. CPC A61K 31/4164; A61Q 17/04; A61K 8/4946; A61Q 19/10; A61Q 19/002; A61K 45/06
USPC 424/59; 514/396

(57) ABSTRACT

Compositions for alleviating mechanically-induced skin inflammation, such as that caused by shaving, contain substituted azole. Methods for alleviating mechanically-induced skin inflammation include the step of applying a composition comprising substituted azole to a skin surface subject to mechanically-induced inflammation. In particular embodiments the substituted azole compound is 1-phenylimidazole, 4-phenylimidazole, or a combination thereof. An effective amount of the composition is applied for a period of time sufficient to alleviate mechanically-induced skin inflammation.

### FIELD OF THE INVENTION

Cosmetic compositions and methods for alleviating mechanically-induced skin inflammation using substituted azole compounds, particularly 1-phenylimidazole, 4-phenylimidazole, or combinations thereof.

### BACKGROUND OF THE INVENTION

The epidermis, the outermost layer of the skin, comprises a cellular continuum of four layers: the stratum corneum, the granular layer, the spinous layer, and the basal layer. Each cellular layer in the epidermis represents various stages along a process in which basal epidermal keratinocytes undergo a continuous cycle of proliferation, differentiation, and apoptosis, moving upward from the basal layer to finally yield corneocytes. These corneocytes form the cornified layer known as the stratum corneum.

Basal keratinocytes reside at the lower portion of the epidermis. These mitotically active cells undergo a proliferative cycle to generate daughter cells that are physically dislocated upward into the spinous and granular layers and undergo the process of differentiation into corneocytes. On passing through the spinous and granular layers, the cells undergo morphological changes that render them flatter in structure as they lose their cellular viability, undergo alternate keratin expression profiles, and transform into cellular remnants. On average, a younger-aged epidermis turns over in about one month, shedding the older cells and replacing them with newer ones, but this process can increase to over forty days in older skin.

The stratum corneum's corneocytes remain connected to one other via proteins and lipids, creating a protective barrier between the organism and its outside environment. This tightly regulated epidermal permeability barrier functions as a physical and selective barrier against chemical and biological insults. Important functions of this barrier include attenuation of the penetration of free radicals and prevention of penetration of harmful radiation, including UV radiation, into deeper layers. The stratum corneum also acts as a permeability barrier and functions to prevent loss of body moisture to the outside environment. Dysfunction of this barrier can lead to chronic skin conditions, diseases, and in extreme cases can even threaten the viability of the organism.

Hair removal is an important part of many individuals' grooming routine and is widely practiced by both males and females. In many cultures, for instance, adult males customarily remove facial hair via shaving on a daily basis (i.e. at least once per day). While frequent shaving maintains a clean-cut appearance, it also results in chronic inflammation of the skin.

As the razor blade moves across the skin's surface to closely cut the hair, the blade also disrupts the skin barrier, prompting activation of the skin's barrier repair process in an effort to restore normal barrier function. Inflammation also results as a consequence of this barrier repair activation (i.e., the wound repair response). With frequent shaving, the barrier is chronically disturbed, the repair process is chronically activated, and chronic inflammation of the stratum corneum results. In addition to the pain, swelling, and redness associated with inflammation, other undesired skin conditions such as dehydration, hypersensitivity, and post-inflammatory hyperpigmentation can result.

The skin's epidermal barrier function is key to its ability to regenerate and protect itself. Accordingly, it would be desirable to provide compositions and methods of treatment that can improve or restore the skin's epidermal barrier functioning when compromised, for example, by mechanically-induced damage such as that caused by shaving. Furthermore, it would also be desirable to alleviate the signs of mechanically-induced chronic inflammation, such as pain, swelling, redness, dehydration, hypersensitivity, and post-inflammatory hyperpigmentation.

### SUMMARY OF THE INVENTION

The present invention provides a method of alleviating mechanically-induced skin inflammation comprising: (a) identifying a skin surface subject to mechanically-induced inflammation; and (b) applying to the skin surface a composition comprising an effective amount of a substituted azole compound represented by the structure:

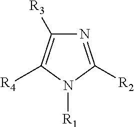

where:
R1: is an alkyl or phenyl electron donating group
R2: is hydrogen
R3 and R4 (which may be identical or different): do not form a fused ring, and each is independently selected from the monodentate group consisting of H, alkyl, and phenyl.

The substituted azole compound can be one or a combination of more than one substituted azole compound. In some embodiments, the substituted azole compound is 1-phenylimidazole:

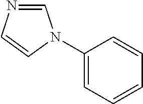 [SMILES: c1ccc(-n2ccnc2)cc1]

In other embodiments, the substituted azole compound is 4-phenylimidazole:

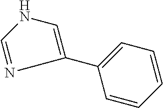 [SMILES: c1ccc(-c2c[nH]cn2)cc1]

In some compositions, the substituted azole compound is a combination of 1-phenylimidazole and 4-phenylimidazole.

The composition comprises an effective amount of the substituted azole compound. In some embodiments, the composition comprises up to 20%, 10%, 5%, 3%, or 1%, and alternatively at least 0.001%, 0.01%, 0.1%. 0.2%, or 0.5%, by weight of the total composition, of the substituted azole compound. Suitable ranges can include any combination of the lower and upper limits, for example from 0.001% to 20%; from 0.001% to 1%; or from 0.5% to 10%, by weight of the composition, of the substituted azole compound. The amounts listed herein are only to be used as a guide, as the optimum amount will depend on the specific substituted azole compound selected, since their potency does vary considerably.

The composition also comprises a dermatologically acceptable carrier. The composition can also include optional ingredients as desired, such as a sunscreen active, an anti-inflammatory agent, and/or a skin tone agent. Exemplary skin tone agents can include vitamin B3 compounds, sugar amines, hexamidine compounds, salicylic acid, and/or 1,3-dihydroxy-4-alkylbenzene.

Alternatively, optional ingredients can be delivered in a second composition that is applied contemporaneously as part of a regimen. In such embodiments, a first composition comprises the substituted azole compound and a second composition comprises desired optional ingredients.

The composition is applied for a period of time sufficient to alleviate mechanically-induced skin inflammation. In particular embodiments, the composition is applied to the neck and/or to a facial skin surface, which may include the perioral, chin, periorbital and/or cheek.

The present invention may take other forms. Further forms of the present invention will be appreciated in the detailed description that follows.

### DETAILED DESCRIPTION OF THE INVENTION

The present invention provides compositions and methods for alleviating mechanically-induced skin inflammation. The method comprises: (a) identifying a skin surface subject to mechanically-induced inflammation; and (b) applying to the skin surface a composition comprising an effective amount of a substituted azole compound for a period of time sufficient to alleviate mechanically-induced skin inflammation.

Unless otherwise indicated expressly or by context, the term “substituted azole” refers to one or more substituted azole compounds represented by the structure set forth herein. A “skin surface subject to mechanically-induced skin inflammation” means a skin surface having one or more signs of mechanically-induced skin inflammation. “Applying” means to apply or spread the composition onto a human skin surface (i.e., epidermis).

As used herein, “alleviating mechanically-induced skin inflammation” is broad enough to include not only minimizing and/or preventing mechanically-induced skin inflammation, but also treating mechanically-induced skin inflammation to effect visually and/or tactilely perceptible positive change or improvement (i.e., benefit) in appearance and/or feel of the signs of mechanically-induced skin inflammation.

“Signs of mechanically-induced skin inflammation” include, but are not limited to, all outwardly visible and/or tactilely perceptible manifestations due to mechanically-induced skin inflammation, such as pain, swelling, redness, dehydration, hypersensitivity, and post-inflammatory hyperpigmentation.

An “effective amount” of a substituted azole compound or of a composition containing such substituted azole compound means an amount of such compound or composition sufficient to significantly (i.e., statistically significant) alleviate mechanically-induced skin inflammation. The particular amount that is effective depends on the specific substituted azole compound selected, since the potency of these compounds does vary.

In some embodiments, the composition comprises up to 20%, 10%, 5%, 3%, or 1%, and alternatively at least 0.001%, 0.01%, 0.1%. 0.2%, or 0.5%, by weight of the total composition, of the substituted azole compound. Suitable ranges can include any combination of the lower and upper limits, for example from 0.001% to 20%; from 0.001% to 1%; or from 0.5% to 10%, by weight of the composition, of the substituted azole compound. These exemplary amounts are only to be used as a guide, as the optimum amount will depend on the potency of the specific substituted azole compound. Hence, the amount of some compounds useful in the present invention may be outside the ranges set forth herein. Determining the effective amount for the chosen substituted azole compound is within the knowledge of one skilled in the art.

The composition further comprises a dermatologically acceptable carrier. The term “dermatologically acceptable” as used herein means that the compositions or components described are suitable for use in contact with human skin tissue without undue toxicity, incompatibility, instability, allergic response, and the like.

The substituted azole compound of the present invention is represented by the structure:

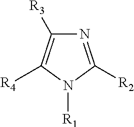

where: R1 is an alkyl or phenyl electron donating group; R2 is hydrogen; R3 and R4 (which may be identical or different) do not form a fused ring and each is independently selected from the monodentate group consisting of H, alkyl, and phenyl. The N-substituted azole compound can be one or a combination of more than one substituted azole compound.

In some embodiments, the substituted azole compound is 1-phenylimidazole:

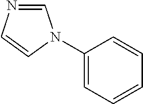 [SMILES: c1ccc(-n2ccnc2)cc1]

In other embodiments, the substituted azole compound is 4-phenylimidazole:

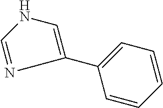 [SMILES: c1ccc(-c2c[nH]cn2)cc1]

In some compositions, the substituted azole compound is a combination of 1-phenylimidazole and 4-phenylimidazole.

### I. Compositions

The present invention relates to various compositions and, more specifically, to compositions for application to a skin surface. The compositions may be in a wide variety of product forms that include, but are not limited to, solutions, suspensions, lotions, creams, gels, toners, sticks, pencil, sprays, aerosols, ointments, cleansing liquid washes and solid bars, shampoos and hair conditioners, pastes, foams, powders, mousses, shaving creams, wipes, strips, patches, electrically-powered patches, wound dressing and adhesive bandages, hydrogels, film-forming products, and facial and skin masks (with and without insoluble sheet). The composition form may follow from the particular dermatologically acceptable carrier chosen, if present in the composition.

For example, when the mechanically-induced inflammation stems from the hair removal process, the composition can be in any form suitable for coming into contact with skin and hair. Non-limiting examples of suitable compositions include cosmetics, moisturizers, lotions, oils, personal cleansers, facial cleansers, shave gels, shave foams, shave oils, after shaves, pre-shave treatments such as lotions, and so forth. The present composition can be used in combination with various hair removal applications (prior to, concurrently with, and/or after), including but not limited to shaving (wet or dry shaving, via electric razors, via powered or manual razors which can be reuseable or disposable, and combinations thereof), epilation, electrolysis, wax or depilatories. In one embodiment, the composition is used as a post-shave moisturizer and/or balm.

A. Substituted Azole Compound

Compositions of the present invention comprise an effective amount of a substituted azole compound represented by the structure:

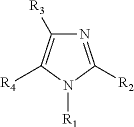

where: R1 is an alkyl or phenyl electron donating group; R2 is hydrogen; R3, and R4 (which may be identical or different) do not form a fused ring and each is independently selected from the monodentate group consisting of H, alkyl, and phenyl.

Electron donating groups have a lone pair of electrons on the atom directly bonded to the ring. The electron donating group increases the aromatic ring's electron density through a resonance donation effect. This is a very stable resonance form, as the resulting carbocation is stabilized by the electron donating group. More stable intermediates (the carbocation) have lower transition state energies and thus a faster reaction rate, resulting in substituents being preferentially directed to positions where they are in conjugation with the aromatic ring. Electron donating groups on an aromatic ring are said to be “activating”, because they increase the rate of the second substitution so that it is higher than that of the standard aromatic molecule.

Applicant has surprisingly discovered that this class of substituted azole compounds can alleviate mechanically-induced skin inflammation when applied topically. Not to be limited by theory, it is believed that the effectiveness of these particular substituted azole compounds is due to their ability to inhibit cytochrome P450 (“CYP”) enzyme activity, and thus function as retinoic acid metabolism blocking agents (“RAMBA”s).

1. Mechanism of Action

Researchers have long appreciated that vitamin A is a critical regulator of growth and differentiation of developing and adult mammalian skin. Vitamin A deficiency causes disruption of normal cellular homeostatic mechanisms, resulting in impairment of skin barrier function. All-trans-retinoic acid (“ATRA”), the biologically most active metabolite of vitamin A, plays a major role in cellular differentiation and proliferation of epithelial tissue.

The main source of retinoic acid (“RA”) in humans, excluding therapeutic dosing, is through synthesis from dietary precursors such as B-carotene and retinyl palmitate. Vitamin A is stored primarily in liver stellate cells as retinyl esters, which are hydrolyzed in hepatocytes by retinyl ester hydrolases to retinol. Retinol, the precursor of RA, is the main circulating retinoid and is obtained from retinol through a two-step synthesis in which the conversion of retinol to retinal is the rate limiting step. Retinol is oxidized intracellularly by retinol dehydrogenases to retinal, and retinal is then metabolized by NAD/NADP-dependent retinal dehydrogenases to RA.

While RA is synthesized endogenously in the body from dietary precursors, it may also be administered exogenously, such as via topical retinoid application. Research has shown that ATRA has the ability to not only prevent but also to repair skin cell damage. With aging, the skin's endogenous ATRA levels are depleted, leading to impaired functioning of the epidermal barrier. Thus, maintaining elevated levels of ATRA is critical in order to maintain barrier function integrity and thus prevent and/or improve the appearance of mechanically-induced skin inflammation.

Unfortunately, therapeutic administration of exogenous (e.g., topical) retinoids has challenges. Although they can be very effective, retinoids are not well tolerated by many people. Common side effects associated with topically applied retinoids include burning and stinging of the skin, peeling, redness, and heightened photosensitivity. Furthermore, the therapeutic effect decreases over time, necessitating the use of increasingly higher retinoid levels to maintain the same level of benefit. Retinoid side effects are largely dose-dependent. As a result, many individuals with a sensitivity to retinoids cannot tolerate levels sufficient to provide the desired positive results. Additionally, retinoids are rapidly metabolized by the body, resulting in the need for higher doses than would otherwise be required to achieve the desired therapeutic effects.

An approach to overcoming the drawbacks associated with exogenous retinoid therapy and/or the rapid elimination of ATRA is to amplify endogenous levels of ATRA by inhibiting the CYP-mediated catabolism of RA using agents known as retinoic acid metabolism blocking agents (RAMBAs). RAMBAs prevent the in vivo catabolism of ATRA by inhibiting the CYP-mediated catabolism enzymes responsible for ATRA elimination Inhibiting the CYP enzymes blocks their metabolism and prolongs RA residence time at the site of action, thus increasing the level of endogenous ATRA within the cells. This results in higher in vivo ATRA concentrations, thus reducing and/or preferably eliminating the need to apply topical exogenous retinoids to achieve the desired effect.

2. In Vitro Cytochrome P450 Inhibition Assay

Cytochrome P450 is a large and diverse group of enzymes that catalyze the oxidation of organic substances. Some members of the CYP family contribute to the elimination of ATRA by catalyzing its 4-hydroxylation in the mammalian liver and skin, including that of humans as well as swine. Applicant evaluated the potential RAMBA activity of several azoles using pig liver microsomes, a rich source of CYP activity, comprising many different CYP 450 isoforms. Therefore, this approach, while a reasonable way to assess CYP inhibitors with broad activities may or may not be the best way to discover RAMBAs with selectivity for the skin, which has a much more narrow complement of CYP expression. As understanding in this area has progressed, a more specific CYP inhibition assay can be used to provide better predictivity of activity in human skin. Nevertheless, this assay may still be used as a general predictor of overall CYP activity.

As shown in Example 2, several compounds were screened through an in vitro CYP assay using pig liver microsomes to determine their effectiveness as CYP inhibitors and correspondingly potential RAMBAs. It is clear from the data in Table 2 that with the exception of the positive control, ketoconazole, none of the tested materials yielded IC₅₀ values at concentrations less than 10 μM. This is likely due to the diversity of CYPs in the microsomal sample, which could dilute the inhibitory activity of CYPs most relevant for ATRA metabolism in the skin.

The metabolism of retinoic acid and vitamin D in the skin is incompletely understood, but there is evidence for the involvement of CYP26A1, CYP3A4 and CYP2C8 in catalyzing the 4-hydroxylation of all-trans RA to 4-hydroxy-RA. It is believed, without being limited by theory, that CYP3A4 may be particularly important for ATRA metabolism in the skin. This is because CYP3A4 is expressed in skin, involved in RA metabolism, a CYP with one of the broadest substrate specificities of all of the known CYPs and available as a human recombinant protein in a commercial kit. Thus, a commercially available CYP3A4 assay was used as a surrogate to predict RAMBA potential in the skin. The method is described in more detail below in Example 3, and the results are illustrated in Table 3. As illustrated in Table 3, the IC₅₀ values of several compounds indicate strong inhibition of CYP3A4 when the IC₅₀ value is ≦10 μM and weak or no inhibition when the IC₅₀ value is >10 μM.

Several simple imidazole structures showed a remarkably high level of inhibitory activity relative to the positive control, ketoconazole. Of particular note are the results for 1-, 2-, and 4-phenylimidazole. These materials are positional isomers, differing from one another only in the location of the phenyl group relative to the imidazole ring. The 1- and 4-phenylimidazole had IC₅₀ values in the same range as climbazole, a known 4-hydroxylase inhibitor that is marketed as an antifungal active. However, the 2-phenylmidazole analogue lacked any significant inhibitory activity, indicating that positioning of the imidazole ring relative to the phenyl group seems to play an important structural component for CYP enzyme interaction. Thus, 1- and 4-phenylimidazole elicit CYP inhibitory activity, while 2-phenylimidazole does not. Accordingly, 1- and 4- can serve as effective RAMBAs, boosting the endogenous ATRA concentration. These results demonstrate that substituted azole compounds having the particular structure set forth herein function differently from other compounds, even when those compounds are positional isomers. (The chemical structures of the materials tested in Example 2 can be found in Table 3 of Example 3. For brevity, the structures are not duplicated by inclusion in both tables.)

3. In Vitro CYP/CYP3A4 Inhibition Assay

The commercially available P450-GLO™ Assay kit (Promega Corporation, Madison Wis.) was used to screen various compounds for potential CYP activity, specifically CYP3A4A inhibition activity. CYP3A4A is thought to be among the primary CYP isoforms responsible for retinoic acid metabolism in the skin.

Three benchmark agents, liarozole, climbazole, and ketoconazole, were assessed for CYP3A4 inhibition to confirm that the inhibition activity (the IC₅₀ for CYP inhibition) measured by the assay corresponded to the activity reported by the published literature.

The results set forth in Table 3 show that the substituted azole compounds having the specific structure set forth herein are CYP inhibitors, and thus function as RAMBAs. Of particular note, once again, are the results for the positional isomers 1-, 2-, and 4-phenylimidazole. Consistent with the results from Example 2's CYP in vitro assay, the R1 substituted 1-phenylimidazole and the R3 substituted 4-phenylimidazole showed inhibitory activity in the CYP3A4 assay, but the R2 substituted 2-phenylimidazole analogue did not. This underscores the importance of the substituted azole structure to the CYP enzyme interaction.

As used in Table 3, “hit” or “no hit” mean, respectively, strong inhibition (IC₅₀ value<10 μM) or weak/no inhibition (IC₅₀ value>10 μM) of CYP3A4.

B. Dermatologically Acceptable Carrier

The compositions of the present invention may also comprise a dermatologically acceptable carrier (which may be referred to as “carrier”) for the composition. The phrase “dermatologically acceptable carrier”, as used herein, means that the carrier is suitable for topical application to the skin surface, has good aesthetic properties, is compatible with the actives in the composition, and will not cause any unreasonable safety or toxicity concerns. In one embodiment, the carrier is present at a level of from 50% to 99%, or from 60% to 98%, or from 70% to 98%, or, alternatively, from 80% to 95%, by weight of the composition.

The carrier can be in a wide variety of forms. Non-limiting examples include simple solutions (e.g., aqueous, organic solvent, or oil based), emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase of the present invention may comprise silicone oils, non-silicone oils such as hydrocarbon oils, esters, ethers, and the like, and mixtures thereof.

The aqueous phase typically comprises water. However, in other embodiments, the aqueous phase may comprise components other than water, including but not limited to water-soluble moisturizing agents, conditioning agents, anti-microbials, humectants and/or other water-soluble skin care actives. In one embodiment, the non-water component of the composition comprises a humectant such as glycerin and/or other polyols. However, it should be recognized that the composition may be substantially (i.e., less than 1% water) or fully anhydrous.

A suitable carrier is selected to yield a desired product form. Furthermore, the solubility or dispersibility of the components (e.g., extracts, sunscreen active, additional components) may dictate the form and character of the carrier. In one embodiment, an oil-in-water or water-in-oil emulsion is preferred. The carrier may further comprise a thickening agent as are well known in the art to provide compositions having a suitable viscosity and rheological character.

Emulsions according to the present invention generally contain a solution as described above and a lipid or oil. Lipids and oils may be derived from animals, plants, or petroleum and may be natural or synthetic. Preferred emulsions also contain a humectant, such as glycerin. Emulsions will preferably further contain from about 0.1% to about 10%, more preferably from about 0.2% to about 5%, of an emulsifier, based on the weight of the composition. Emulsifiers may be nonionic, anionic or cationic. The emulsifier can be a polymer, a surfactant or a mixture thereof. Suitable emulsifiers are disclosed in, for example, U.S. Pat. Nos. 3,755,560, 4,421,769, and McCutcheon's Detergents and Emulsifiers, North American Edition, pages 317-324 (1986).

a. Water-in-Oil Emulsion

Water in oil emulsions are characterized as having a continuous hydrophobic, water insoluble oil phase and a water phase dispersed therein. The “oil phase” can contain oil, silicone or mixtures thereof. The distinction of whether the emulsion is characterized as a water-in-oil or water-in-silicone emulsion is a function of whether the oil phase is composed of primarily oil or silicone. A preferred example of a water-in-silicone emulsion is described below.

1. Continuous Silicone Phase

Preferred water-in-silicone emulsions of the present invention comprise from about 1% to about 60%, preferably from about 5% to about 40%, more preferably from about 10% to about 30%, by weight of a continuous silicone phase. The continuous silicone phase exists as an external phase that contains or surrounds the discontinuous aqueous phase described hereinafter.

The continuous silicone phase contains a silicone elastomer and/or polyorganosiloxane oil. The continuous silicone phase of these preferred emulsions comprises between about 50% and about 99.9% by weight of organopolysiloxane oil and less than about 50% by weight of a non-silicone oil. In a preferred embodiment, the continuous silicone phase comprises at least about 50%, preferably from about 60% to about 99.9%, more preferably from about 70% to about 99.9%, and even more preferably from about 80% to about 99.9%, polyorganosiloxane oil by weight of the continuous silicone phase, and up to about 50% non-silicone oils, preferably less about 40%, more preferably less than about 30%, even more preferably less than about 10%, and still more preferably less than about 2%, by weight of the continuous silicone phase.

2. Polyorganopolysiloxane Oil

The organopolysiloxane oil for use in the composition may be volatile, non-volatile, or a mixture of volatile and non-volatile silicones. The term “nonvolatile” as used in this context refers to those silicones that are liquid under ambient conditions and have a flash point (under one atmospheric of pressure) of or greater than about 100° C. The term “volatile” as used in this context refers to all other silicone oils. Suitable organopolysiloxanes can be selected from a wide variety of silicones spanning a broad range of volatilities and viscosities. Examples of suitable organopolysiloxane oils include polyalkylsiloxanes, cyclic polyalkylsiloxanes, and polyalkylarylsiloxanes.

Suitable polyalkylsiloxanes include polyalkylsiloxanes with viscosities of from about 0.5 to about 1,000,000 centistokes at 25° C. Commercially available polyalkylsiloxanes include polydimethylsiloxanes, which are also known as dimethicones, examples of which include the Vicasil® series sold by General Electric Company and the Dow Corning® 200 series sold by Dow Corning Corporation. Cyclic polyalkylsiloxanes suitable for use in the composition include those commercially available such as Dow Corning® 244, Dow Corning® 344 fluid, and Dow Corning® 345 fluid.

Also useful are materials such as trimethylsiloxysilicate, which is a polymeric material corresponding to the general chemical formula [(CH₂)₃SiO_{1/2}]ₓ[SiO₂]y, wherein x is an integer of from about 1 to about 500 and y is an integer of from about 1 to about 500. A commercially available trimethylsiloxysilicate is sold as a mixture with dimethicone as Dow Corning® 593 fluid.

Dimethiconols are also suitable for use in the composition. These compounds can be represented by the chemical formulas R₃SiO[R₂SiO]ₓSiR₂OH and HOR₂SiO[R₂SiO]ₓSiR₂OH wherein R is an alkyl group (preferably R is methyl or ethyl) and x is an integer of from 0 to about 500, chosen to achieve the desired molecular weight. Commercially available dimethiconols are typically sold as mixtures with dimethicone or cyclomethicone (e.g. Dow Corning® 1401, 1402, and 1403 fluids).

Polyalkylaryl siloxanes are also suitable for use in the composition, particularly those having viscosities of from about 15 to about 65 centistokes at 25° C.

Preferred for use herein are organopolysiloxanes selected from the group consisting of polyalkylsiloxanes, alkyl substituted dimethicones, cyclomethicones, trimethylsiloxysilicates, dimethiconols, polyalkylaryl siloxanes, and mixtures thereof. More preferred for use herein are polyalkylsiloxanes and cyclomethicones. Preferred among the polyalkylsiloxanes are dimethicones.

As stated above, the continuous silicone phase may contain one or more non-silicone oils. Suitable non-silicone oils have a melting point of about 25° C. or less under about one atmosphere of pressure. Examples of non-silicone oils suitable for use in the continuous silicone phase are those well known in the chemical arts in topical personal care products which can be in the form of emulsions, e.g., mineral oil, vegetable oils, synthetic oils, semisynthetic oils, fatty acid esters, etc.

3. Silicone Elastomer

The compositions of the present invention also include from about 0.1% to about 30%, by weight of the composition, of a silicone elastomer component. Preferably, the composition includes from about 1% to about 30%, more preferably from about 2% to about 20%, by weight of the composition, of the silicone elastomer component.

Suitable for use herein are silicone elastomers, which can be emulsifying or non-emulsifying crosslinked siloxane elastomers or mixtures thereof. No specific restriction exists as to the type of curable organopolysiloxane composition that can serve as starting material for the crosslinked organopolysiloxane elastomer. Examples in this respect are addition reaction-curing organopolysiloxane compositions which cure under platinum metal catalysis by the addition reaction between SiH-containing diorganopolysiloxane and organopolysiloxane having silicon-bonded vinyl groups; condensation-curing organopolysiloxane compositions which cure in the presence of an organotin compound by a dehydrogenation reaction between hydroxyl-terminated diorganopolysiloxane and SiH-containing diorganopolysiloxane and condensation-curing organopolysiloxane compositions which cure in the presence of an organotin compound or a titanate ester,

Addition reaction-curing organopolysiloxane compositions are preferred for their rapid curing rates and excellent uniformity of curing. A particularly preferred addition reaction-curing organopolysiloxane composition is prepared from:
(A) an organopolysiloxane having at least 2 lower alkenyl groups in each molecule;
(B) an organopolysiloxane having at least 2 silicon-bonded hydrogen atoms in each molecule; and
(C) a platinum-type catalyst.

In one embodiment the composition includes an emulsifying crosslinked organopolysiloxane elastomer, a non-emulsifying crosslinked organopolysiloxane elastomer, or a mixture thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit. Preferred emulsifying elastomers herein include polyoxyalkylene modified elastomers formed from divinyl compounds, particularly siloxane polymers with at least two free vinyl groups, reacting with Si—H linkages on a polysiloxane backbone. Preferably, the elastomers are dimethyl polysiloxanes crosslinked by Si—H sites on a molecularly spherical MQ resin. Emulsifying crosslinked organopolysiloxane elastomers can notably be chosen from the crosslinked polymers described in U.S. Pat. Nos. 5,412,004, 5,837,793, and 5,811,487. An emulsifying elastomer comprising dimethicone copolyol crosspolymer (and) dimethicone is available from Shin Etsu under the tradename KSG-21.

Advantageously, the non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. No. 4,970,252, U.S. Pat. No. 5,760,116, and U.S. Pat. No. 5,654,362. Additional crosslinked organopolysiloxane elastomers useful in the present invention are disclosed in Japanese Patent Application JP 61-18708, assigned to Pola Kasei Kogyo KK.

Commercially available elastomers preferred for use herein are Dow Corning's 9040 silicone elastomer blend, Shin Etsu's KSG-21, and mixtures thereof.

4. Carrier for Silicone Elastomer

The topical compositions of the present invention include from about 1% to about 80%, by weight of the composition, of a suitable carrier for the for the crosslinked organopolysiloxane elastomer component described above. The carrier, when combined with the cross-linked organopolysiloxane elastomer particles of the present invention, serves to suspend and swell the elastomer particles to provide an elastic, gel-like network or matrix. The carrier for the cross-linked siloxane elastomer is liquid under ambient conditions, and preferably has a low viscosity to provide for improved spreading on skin.

Concentrations of the carrier in the cosmetic compositions of the present invention will vary primarily with the type and amount of carrier and the cross-linked siloxane elastomer employed. Preferred concentrations of the carrier are from about 5% to about 50%, more preferably from about 5% to about 40%, by weight of the composition.

The carrier for the cross-linked siloxane elastomer includes one or more liquid carriers suitable for topical application to human skin. These liquid carriers may be organic, silicone-containing or fluorine-containing, volatile or non-volatile, polar or non-polar, provided that the liquid carrier forms a solution or other homogenous liquid or liquid dispersion with the selected cross-linked siloxane elastomer at the selected siloxane elastomer concentration at a temperature of from about 28° C. to about 250° C., preferably from about 28° C. to about 100° C., preferably from about 28° C. to about 78° C. The term “volatile” as used herein refers to all materials that are not “non-volatile” as previously defined herein. The phrase “relatively polar” as used herein means more polar than another material in terms of solubility parameter; i.e., the higher the solubility parameter the more polar the liquid. The term “non-polar” typically means that the material has a solubility parameter below about 6.5 (cal/cm³)^{0.5}.

5. Non-Polar, Volatile Oils

The non-polar, volatile oil tends to impart highly desirable aesthetic properties to the compositions of the present invention. Consequently, the non-polar, volatile oils are preferably utilized at a fairly high level. Non-polar, volatile oils particularly useful in the present invention are silicone oils; hydrocarbons; and mixtures thereof. Such non-polar, volatile oils are disclosed, for example, in Cosmetics, Science, and Technology, Vol. 1, 27-104 edited by Balsam and Sagarin, 1972. Examples of preferred non-polar, volatile hydrocarbons include polydecanes such as isododecane and isodecane (e.g., Permethyl-99A which is available from Presperse Inc.) and the C7-C8 through C12-C15 isoparaffins (such as the Isopar Series available from Exxon Chemicals). Particularly preferred volatile silicone oils are selected from cyclic volatile silicones with formula:

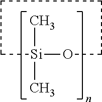

wherein n is from about 3 to about 7; and linear volatile silicones with formula:

(CH₃)₃Si—O—[Si(CH₃)₂—O]ₘ—Si(CH₃)₃

wherein m is from about 1 to about 7. Linear volatile silicones generally have a viscosity of less than about 5 centistokes at 25° C., whereas the cyclic silicones have viscosities of less than about 10 centistokes at 25° C. Highly preferred examples of volatile silicone oils include cyclomethicones of varying viscosities, e.g., Dow Corning 200, Dow Corning 244, Dow Corning 245, Dow Corning 344, and Dow Corning 345, (from Dow Corning Corp.); SF-1204 and SF-1202 Silicone Fluids (from G.E. Silicones), GE 7207 and 7158 (from General Electric Co.); and SWS-03314 (from SWS Silicones Corp.).

6. Relatively Polar, Non-Volatile Oils

The non-volatile oil is “relatively polar” as compared to the non-polar, volatile oil discussed above. Therefore, the non-volatile co-carrier is more polar (i.e., has a higher solubility parameter) than at least one of the non-polar, volatile oils. Relatively polar, non-volatile oils potentially useful in the present invention are disclosed, for example, in Cosmetics, Science, and Technology, Vol. 1, 27-104 edited by Balsam and Sagarin, 1972; U.S. Pat. Nos. 4,202,879 and 4,816,261. Relatively polar, non-volatile oils useful in the present invention are preferably selected from silicone oils; hydrocarbon oils; fatty alcohols; fatty acids; esters of mono and dibasic carboxylic acids with mono and polyhydric alcohols; polyoxyethylenes; polyoxypropylenes; mixtures of polyoxyethylene and polyoxypropylene ethers of fatty alcohols; and mixtures thereof.

7. Non-Polar, Non-Volatile Oils

In addition to the liquids discussed above, the carrier for the cross-linked siloxane elastomer may optionally include non-volatile, non-polar oils. Typical non-volatile, non-polar emollients are disclosed, for example, in Cosmetics, Science, and Technology, Vol. 1, 27-104 edited by Balsam and Sagarin, 1972; U.S. Pat. Nos. 4,202,879 and 4,816,261. The non-volatile oils useful in the present invention are essentially non-volatile polysiloxanes, paraffinic hydrocarbon oils, and mixtures thereof.

8. Dispersed Aqueous Phase

The compositions of the present invention can comprise from about 30% to about 90%, more preferably from about 50% to about 85%, and even more preferably from about 70% to about 80% of a dispersed aqueous phase. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The dispersed aqueous phase is a dispersion of small aqueous particles or droplets suspended in and surrounded by the continuous silicone phase described hereinbefore.

The aqueous phase can be water, or a combination of water and one or more water soluble or dispersible ingredients. Nonlimiting examples of such optional ingredients include thickeners, acids, bases, salts, chelants, gums, water-soluble or dispersible alcohols and polyols, buffers, preservatives, sunscreening agents, colorings, and the like.

The topical compositions of the present invention will typically comprise from about 25% to about 90%, preferably from about 40% to about 85%, more preferably from about 60% to about 80%, water in the dispersed aqueous phase by weight.

9. Emulsifier for Dispersing the Aqueous Phase

The water-in-silicone emulsions of the present invention preferably comprise an emulsifier. In one embodiment, the composition contains from about 0.1% to about 10% emulsifier, more preferably from about 0.2% to about 7.5%, even more preferably from about 0.5% to about 5%, emulsifier by weight of the composition. The emulsifier helps disperse and suspend the aqueous phase within the continuous silicone phase.

A wide variety of emulsifying agents can be employed herein to form the preferred water-in-silicone emulsion. Known or conventional emulsifying agents can be used in the composition, provided that the selected emulsifying agent is chemically and physically compatible with essential components of the composition, and provides the desired dispersion characteristics. Suitable emulsifiers include silicone emulsifiers, non-silicon-containing emulsifiers, and mixtures thereof, known by those skilled in the art for use in topical personal care products. Preferably these emulsifiers have an HLB value of less than about 14, more preferably from about 2 to about 14, and even more preferably from about 4 to about 14. Emulsifiers having an HLB value outside of these ranges can be used in combination with other emulsifiers to achieve an effective weighted average HLB for the combination that falls within these ranges.

Silicone emulsifiers are preferred. A wide variety of silicone emulsifiers are useful herein. These silicone emulsifiers are typically organically modified organopolysiloxanes, also known to those skilled in the art as silicone surfactants. Useful silicone emulsifiers include dimethicone copolyols.

Nonlimiting examples of dimethicone copolyols and other silicone surfactants useful as emulsifiers herein include polydimethylsiloxane polyether copolymers with pendant polyethylene oxide side chains, polydimethylsiloxane polyether copolymers with pendant polypropylene oxide side chains, polydimethylsiloxane polyether copolymers with pendant mixed polyethylene oxide and polypropylene oxide side chains, polydimethylsiloxane polyether copolymers with pendant mixed poly(ethylene)(propylene)oxide side chains, polydimethylsiloxane polyether copolymers with pendant organobetaine side chains, polydimethylsiloxane polyether copolymers with pendant carboxylate side chains, polydimethylsiloxane polyether copolymers with pendant quaternary ammonium side chains; and also further modifications of the preceding copolymers containing pendant C2-C30 straight, branched, or cyclic alkyl moieties. Examples of commercially available dimethicone copolyols useful herein sold by Dow Corning Corporation are Dow Corning® 190, 193, Q2-5220, 2501 Wax, 2-5324 fluid, and 3225C (this latter material being sold as a mixture with cyclomethicone). Cetyl dimethicone copolyol is commercially available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Other nonlimiting examples of dimethicone copolyols also include lauryl dimethicone copolyol, dimethicone copolyol acetate, diemethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, and dimethicone copolyol stearate.

Among the non-silicone-containing emulsifiers useful herein are various non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof. Other suitable emulsifiers are described, for example, in McCutcheon's, Detergents and Emulsifiers, North American Edition (1986), published by Allured Publishing Corporation; U.S. Pat. Nos. 5,011,681, 4,421,769, and 3,755,560.

b. Oil-in-Water Emulsions

Other preferred topical carriers include oil-in-water emulsions, having a continuous aqueous phase and a hydrophobic, water-insoluble phase (“oil phase”) dispersed therein. The “oil phase” can contain oil, silicone or mixtures thereof, and includes but is not limited to the oils and silicones described above in the section on water-in-oil emulsions. The distinction of whether the emulsion is characterized as an oil-in-water or silicone-in-water emulsions is a function of whether the oil phase is composed of primarily oil or silicone. The water phase of these emulsions consists primarily of water, but can also contain various other ingredients such as those water phase ingredients listed in the above section on water-in-oil emulsion. The preferred oil-in-water emulsions comprises from about 25% to about 98%, preferably from about 65% to about 95%, more preferably from about 70% to about 90% water by weight of the total composition.

In addition to a continuous water phase and dispersed oil or silicone phase, these oil-in-water compositions also comprise an emulsifier to stabilize the emulsion. Emulsifiers useful herein are well known in the art, and include nonionic, anionic, cationic, and amphoteric emulsifiers. Non-limiting examples of emulsifiers useful in the oil-in-water emulsions of this invention are given in McCutcheon's, Detergents and Emulsifiers, North American Edition (1986), published by Allured Publishing Corporation; U.S. Pat. No. 5,011,681; U.S. Pat. No. 4,421,769; and U.S. Pat. No. 3,755,560.

C. Skin Tone Agent

In some embodiments, it may be desirable to include a skin tone agent in the composition. The skin tone agents can be included to further improve overall skin tone. When present, the compositions of the present invention can contain up to 50%, 40%, 30%, 20%, 10%, 5%, or 3%, by weight of the composition, of the skin tone agent. When present, the compositions of the present invention can contain at least 0.001%, 0.01%, 0.1%, 0.2%, 0.5%, or 1%, by weight of the composition, of the skin tone agent. Suitable ranges include any combination of the lower and upper limits including suitable ranges from 0.1% to 50%; from 0.2% to 20%; or from 1% to 10%, by weight of the composition, of the skin tone agent. The amounts listed herein are only to be used as a guide, as the optimum amount of the skin tone agent will depend on the specific active selected since their potency does vary considerably.

Suitable skin tone agents include, but are not limited to, sugar amines, vitamin B3 compounds, arbutin, deoxyarbutin, 1,3-dihydroxy-4-alkylbenzene such as hexylresorcinol, sucrose dilaurante, bakuchoil (4-[(1E,3S)-3-ethenyl-3,7-dimethyl-1,6 octadienyl]phenol or monterpene phenol), pyrenoine (available from Biotech Marine, France), panicum miliaceum seed extract, arlatone dioic acid, cinnamic acid, ferulic acid, achromaxyl, methyl nicotinamide, oil soluble licorice extract, folic acid, undecylenic acid (i.e., undecenoic acid), zinc undecylenate, thiamine (Vitamin B 1) and its hydrochloride, L-tryptophan, helianthus annulus (sunflower) and vitis vinifera (grape) leaf extract, carnosine (i.e., dragosine), methyl gentisate, 1,2-hexandiol and 1,2-octandiol (i.e., combination sold as Symdiol 68 by Symrise AG, Germany), inositol, decylenoylphenylalanine (e.g., sold under the tradename Sepiwhite by Seppic, France), kojic acid, hexamidine compounds, and salicylic acid.

In certain embodiments, the skin tone agent is selected from vitamin B3 compounds, sugar amines, hexamidine compounds, salicylic acid, and a 1,3-dihydroxy-4-alkylbenzene such as hexylresorcinol. As used herein, “vitamin B₃ compound” means a compound having the formula:

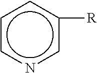

wherein R is —CONH₂ (i.e., niacinamide), —COOH (i.e., nicotinic acid) or —CH₂OH (i.e., nicotinyl alcohol); derivatives thereof; and salts of any of the foregoing. As used herein, “sugar amine” includes isomers and tautomers of such and its salts (e.g., HCl salt) and its derivatives. Examples of sugar amines include glucosamine, N-acetyl glucosamine, mannosamine, N-acetyl mannosamine, galactosamine, N-acetyl galactosamine, their isomers (e.g., stereoisomers), and their salts (e.g., HCl salt). As used herein, “hexaminide compound” means a compound having the formula:

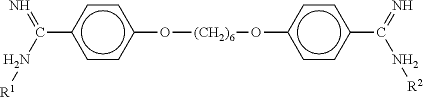

wherein R¹ and R² are optional or are organic acids (e.g., sulfonic acids, etc.). In one embodiment, the hexamidine compound is hexamidine diisethionate.

Furthermore, the skin tone agent of the present invention can include a xanthine compound. As used herein, “xanthine compound” means one or more xanthines, derivatives thereof, and mixtures thereof. Xanthine compounds that can be useful herein include, but are not limited to, caffeine, xanthine, 1-methyl xanthine, theophylline, theobromine, derivatives thereof, and mixtures thereof. In one embodiment, the composition comprises from about 0.1% to about 10% of a xanthine compound, in another embodiment from about 0.5% to about 5% of a xanthine compound, and in yet another embodiment from about 1% to about 2% of a xanthine compound.

D. Anti-Inflammatory Agents

Hyperpigmentation may result from skin inflammation. Transient inflammatory events triggering hyperpigmentation and, more specifically, post-inflammatory hyperpigmentation include, but are not limited to, acne lesions, ingrown hairs, scratches, insect bites, surfactant damage, allergens, and short-term UV exposure. Inflammation induced hyperpigmentation including post-inflammatory hyperpigmentation may be managed by incorporating into the compositions of the present invention an anti-inflammatory agent. When present, the compositions of the present invention can contain up to 20%, 10%, 5%, 3%, or 1% by weight of the composition, of the anti-inflammatory agent. When present, the compositions of the present invention can contain at least 0.001%, 0.01%, 0.1%, 0.2%, 0.3%, 0.5%, or 1%, by weight of the composition, of the anti-inflammatory agent. Suitable ranges include any combination of the lower and upper limits Exact amounts will vary depending upon the chosen anti-inflammatory agent; determining the appropriate amount is within the knowledge of one of skilled in the art.

Suitable anti-inflammatory agents include, but are not limited to nonsteroidal anti-inflammatory agents (“NSAIDS” including but not limited to ibuprofen, naproxen, flufenamic acid, etofenamate, aspirin, mefenamic acid, meclofenamic acid, piroxicam and felbinac), glycyrrhizic acid (also known as glycyrrhizin, glycyrrhixinic acid, and glycyrrhetinic acid glycoside) and salts such as dipotassium glycyrrhizate, glycyrrhetenic acid, licorice extracts, bisabolol (e.g., alpha bisabolol), manjistha (extracted from plants in the genus Rubia, particularly Rubia cordifolia), and guggal (extracted from plants in the genus Commiphora, particularly Commiphora mukul), kola extract, chamomile, and sea whip extract (extracts from plant in the order Gorgonacea), derivatives of any of the foregoing, and mixtures thereof.

E. Sunscreen Actives

The compositions of the subject invention may comprise one or more sunscreen actives (or sunscreen agents) and/or ultraviolet light absorbers. As used herein, “sunscreen active” collectively includes sunscreen actives, sunscreen agents, and/or ultraviolet light absorbers. Sunscreen actives include both sunscreen agents and physical sunblocks. Sunscreen actives may be organic or inorganic. Examples of suitable sunscreen actives are disclosed in Personal Care Product Council's International Cosmetic Ingredient Dictionary and Handbook, Thirteenth Edition, as “sunscreen agents.”

Suitable sunscreen actives include 2-ethylhexyl-p-methoxycinnamate (commercially available as PARSOL™ MCX), 4,4′-t-butyl methoxydibenzoyl-methane (commercially available as PARSOL™ 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, menthyl anthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, benzylidene camphor and derivatives thereof, titanium dioxide, and mixtures thereof.

When present, the compositions of the present invention can contain up to 20%, 10%, 5%, 3%, or 1% by weight of the composition, of the sunscreen active. When present, the compositions of the present invention can contain at least 0.001%, 0.01%, 0.1%, 0.2%, 0.3%, 0.5%, or 1%, by weight of the composition, of the sunscreen active. Suitable ranges include any combination of the lower and upper limits Exact amounts will vary depending upon the chosen sunscreen active and the desired Sun Protection Factor (SPF), which is within the knowledge of one of skilled in the art.

F. Lathering Surfactants

Where the composition is a wash or cleansing composition, the carrier can comprise one or more lathering surfactants and the carrier can be at a level of from about 60% to about 99.99%. A lathering surfactant defined herein as surfactant, which when combined with water and mechanically agitated generates a foam or lather. Preferably, these surfactants or combinations of surfactants should be mild, which means that these surfactants provide sufficient cleansing or detersive benefits but do not overly dry the skin or hair while still lathering. Those of skill in the art should understand that the lathering surfactant is in addition to the surfactant derived from a predominantly unsaturated triglyceride described above.

A wide variety of lathering surfactants are useful herein and include those selected from the group consisting of anionic lathering surfactants, nonionic lather surfactants, amphoteric lathering surfactants, and mixtures thereof. Generally, the lathering surfactants are fairly water soluble. When used in the composition, at least about 4% of the lathering surfactants have a HLB value greater than about ten. Examples of such surfactants are found in and U.S. Pat. No. 5,624,666. Cationic surfactants can also be used as optional components, provided they do not negatively impact the overall lathering characteristics of the required lathering surfactants

Concentrations of these surfactant are from about 10% to about 20%, alternatively from about 6% to about 25%, and alternatively from about 4% to about 30% by weight of the composition. To avoid skin irritation issues, the compositions should have a ratio by weight of the composition of anionic surfactant to amphoteric and/or zwitterionic surfactant is from about 1.1:1 to about 1:1.5, alternatively from about 1.25:1 to about 1:2, and alternatively from about 1.5:1 to about 1:3.

Anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. A wide variety of anionic lathering surfactants are useful herein. Non-limiting examples of anionic lathering surfactants include those selected from the group consisting of sarcosinates, sulfates, sulfonates, isethionates, taurates, phosphates, lactylates, glutamates, and mixtures thereof.

Other anionic materials useful herein are soaps (i.e., alkali metal salts, e.g., sodium or potassium salts) of fatty acids, typically having from about 8 to about 24 carbon atoms, preferably from about 10 to about 20 carbon atoms, monoalkyl, dialkyl, and trialkylphosphate salts, alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and alkanolamine (e.g., triethanolamine). Also useful are taurates which are based on taurine, which is also known as 2-aminoethanesulfonic acid, and glutamates, especially those having carbon chains between C₈ and C₁₆.

Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Suitable amphoteric or zwitterionic detersive surfactants for use in the compositions herein include those which are known for use in hair care or other personal care cleansing. Concentration of such amphoteric detersive surfactants is from about 1% to about 10%, alternatively from about 0.5% to about 20% by weight of the composition. Non-limiting examples of suitable zwitterionic or amphoteric surfactants are described in U.S. Pat. No. 5,104,646 and U.S. Pat. No. 5,106,609.

Nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety. Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyldi(2-hydroxyethyl)amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Preferred lathering surfactants for use herein are the following, wherein the anionic lathering surfactant is selected from the group consisting of ammonium lauroyl sarcosinate, sodium trideceth sulfate, sodium lauroyl sarcosinate, sodium myristoyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isethionate, sodium cetyl sulfate, sodium lauroyl lactylate, triethanolamine lauroyl lactylate, and mixtures thereof; wherein the nonionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

G. Other Optional Components

The compositions of the present invention may optionally contain a variety of other ingredients provided that they do not unacceptably alter the benefits of the invention. When present, compositions of the present invention may contain from 0.0001% to 50%; from 0.001% to 20%; or, alternately, from 0.01% to 10%, by weight of the composition, of the optional components. The amounts listed herein are only to be used as a guide, as the optimum amount of the optional components used in a composition will depend on the specific active selected since their potency does vary considerably. Hence, the amount of some optional components useful in the present invention may be outside the ranges listed herein.

The optional components, when incorporated into the composition, should be suitable for use in contact with human skin tissue without undue toxicity, incompatibility, instability, allergic response, and the like. The compositions of the present invention may include optional components such as anti-acne actives, desquamation actives, anti-cellulite agents, chelating agents, flavonoids, tanning active, non-vitamin antioxidants and radical scavengers, hair growth regulators, anti-wrinkle actives, anti-atrophy actives, minerals, phytosterols and/or plant hormones, N-acyl amino acid compounds, antimicrobial or antifungal actives, and other useful skin care actives, which are described in further detail in U.S. application publication No. US2006/0275237A1 and US2004/0175347A1.

The Personal Care Product Council's International Cosmetic Ingredient Dictionary and Handbook, Thirteenth Edition, describes a wide variety of non-limiting cosmetic and pharmaceutical ingredients commonly used in the skin care industry, which are suitable optional components for use in the compositions of the present invention. Examples of these ingredient classes include: abrasives, absorbents, aesthetic components such as fragrances, pigments, colorings/colorants, essential oils, anti-caking agents, antifoaming agents, antimicrobials, binders, biological additives, buffering agents, bulking agents, chelating agents, chemical additives, colorants, cosmetic astringents, cosmetic biocides, denaturants, drug astringents, emollients, external analgesics, film formers or materials, opacifying agents, pH adjusters, preservatives, propellants, reducing agents, sequestrants, skin cooling agents, skin protectants, thickeners viscosity modifiers, vitamins, and combinations thereof.

### II. Methods of Treatment

Various methods of treatment, application, regulation, or improvement may utilize the aforementioned compositions. Identification of a region of skin subject to mechanically-induced inflammation may occur on any relevant skin surface of the body that has been or will be shaved. Skin surfaces of the most concern include those such as facial and neck skin surfaces, foot and leg skin surfaces, bikini area skin surfaces, underarm skin surfaces, and cranial skin surfaces. In particular, identification of the region of mechanically-induced skin inflammation may be on a facial skin surface including the perioral, chin, periorbital, neck, and/or cheek skin surfaces.

One suitable method of alleviating mechanically-induced skin inflammation includes the step of topically applying a composition comprising an effective amount of substituted azole to the skin surface, wherein the composition is applied for a period of time sufficient to improve the appearance of the mechanically-induced skin inflammation. For example, the composition can be applied before, during, and/or after shaving.

The method may comprise the step of applying the composition to the previously identified area of skin subject to mechanically-induced skin inflammation, and/or an area where one seeks to prevent mechanically-induced skin inflammation. Many regimens exist for the application of the composition. The composition may be applied at least once a day, twice a day, or on a more frequent daily basis, during a treatment period. For example, a composition in the form of a shaving cream or gel can be applied to a skin surface prior to shaving; after shaving, the composition in the form of a leave-on skin cream can be applied to the shaved skin area.

Treatment frequency typically coincides with the frequency of shaving, although if desired one can also apply the composition in suitable form between shaving events. Thus, it is anticipated that in many cases the composition will be applied at least once daily in order to alleviate the mechanically-induced skin inflammation caused by shaving. In some cases, the composition will be applied more than once per day, for instance in the morning after shaving and then again later in the day (e.g., the user applies the composition a second time but does not also shave the skin a second time that day).

The treatment period is of sufficient time to provide alleviation of mechanically-induced skin inflammation. Typically, the composition is applied at least one time per each shaving event, however it can be applied less frequently if desired. The treatment period may be at least 1 week, and in some embodiments the treatment period may last 4 weeks or 8 weeks. In certain embodiments, the treatment period will extend over multiple months (i.e., 3-12 months) or multiple years. In one embodiment the composition is applied at least once a day during a treatment period of at least 4 weeks or at least 8 weeks. In one embodiment the composition is applied twice a day during a treatment period of at least 4 weeks or 8 weeks.

The step of applying the composition may be accomplished by localized application. In reference to application of the composition, the terms “localized”, “local”, or “locally” mean that the composition is delivered to the targeted area (e.g., the face or legs) while minimizing delivery to skin surfaces not requiring treatment. The composition may be applied and lightly massaged into an area subject to mechanically-induced skin inflammation. The form of the composition or the dermatologically acceptable carrier should be selected to facilitate localized application. While certain embodiments of the present invention contemplate applying a composition locally to an area (e.g., when in the form of shaving cream), it will be appreciated that compositions of the present invention can be applied more generally or broadly to one or more skin surfaces (e.g., when in the form of a leave-on skin cream). In some embodiments, the composition may be delivered by a variety of applicators appropriate for localized and general application.

While some methods described herein contemplate applying the compositions of the present invention with an applicator, it will be appreciated that applicators are not required and the compositions of the present invention can also be applied directly by using one's finger or in other conventional manners.

In one embodiment, the method comprises the steps of applying a first composition comprising an effective amount of substituted azole to a skin surface and of applying a second composition to the skin surface, before or after the first composition. The first and second compositions may be any compositions described herein; however, the second composition may optionally comprise an effective amount of the substituted azole compound present in the first composition. The second composition may comprise one or more skin tone agents, sunscreen actives, anti-inflammatory agents, or other optional components. The first composition may be generally or locally applied, while the second composition may be generally or locally applied to the skin surface including the mechanically-induced skin inflammation to which the first composition is applied. In certain embodiments, the skin surface is facial skin surface which includes one or more of the perioral, chin, periorbital, nose, and cheek skin surfaces. In another embodiment, the first and second compositions are applied contemporaneously to at least the cheek, forehead, and chin/perioral skin surfaces. For general application to a skin surface and, particularly a facial skin surface, the dosed amount of the first or second composition may be between about 1 to about 50 μL/cm² per application (i.e., per single application to the skin surfaces).

Suitable methods may comprise any one or more of the abovementioned steps. All of the aforementioned steps are applicable to alleviating mechanically-induced skin inflammation.

### EXAMPLES

Below are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minor materials will vary depending on the physical and chemical characteristics of the particular ingredients selected to make the present invention as described herein. All example compositions may be used to alleviate mechanically-induced skin inflammation.

### Example 1

### Exemplary Compositions

### Example Set A

### Moisturizer/Balm Making Instructions

Phase A materials are combined and heated in a container. Phase B materials are combined and heated in a separate container. Phase B is added to Phase A under high shear. The mixture of Phases A and B is cooled and the contents of Phase C are added with mixing. Phase D materials are blended in a separate container and added to the mixture of Phases A, B, and C. The final mixture is stirred until well blended. Qs means quantity sufficient to reach 100%.

TABLE 1A
Moisturizer/Balm Ingredients

| | Sample | Sample | Sample | Sample | Sample | Sample |
|---|---|---|---|---|---|---|
| | 1 | 2 | 3 | 4 | 5 | 6 |
| Phase A | | | | | | |
| Water | Qs | Qs | Qs | Qs | Qs | Qs |
| Glycerin | 8.0000% | 6.0000% | 6.0000% | 6.0000% | 4.0000% | 5.0000% |
| Sorbitol | | | | | 2.0000% | 2.0000% |
| Disodium EDTA | 0.1000% | 0.1000% | 0.1000% | 0.1000% | 0.1000% | 0.1000% |
| Phase B | | | | | | |
| Cetearyl Alcohol | | | | | | |
| Emulgade Pl 68/50 ¹ | 0.2000% | 0.2000% | 0.2000% | 0.2000% | 0.2000% | 0.2000% |
| Cetiol SN ² | 5.0000% | 5.0000% | 5.0000% | 3.5000% | 3.5000% | 5.0000% |
| Cetyl Alcohol | 0.8900% | 0.8900% | 0.8900% | 0.8900% | 0.8900% | 0.8900% |
| PEG-100 Stearate | 0.1000% | 0.1000% | 0.1000% | | 0.1000% | 0.1000% |
| Polymethylsilsesquioxane | 1.0000% | | 1.0000% | 1.0000% | 1.0000% | |
| Sorbitan Stearate | 0.1000% | 0.1000% | 0.1000% | 0.1000% | 0.1000% | 0.1000% |
| Steareth-2 | 0.1000% | 0.1000% | | | | |
| Steareth-21 | 0.1000% | 0.1000% | | | | |
| Stearic acid | 0.1000% | 0.1000% | 0.1000% | | 0.1000% | 0.1000% |
| Stearyl Alcohol | 0.6100% | 0.6100% | 0.6100% | 0.6100% | 0.6100% | 0.6100% |
| Phase C | | | | | | |
| Aluminum Starch | 4.0000% | 4.0000% | 4.0000% | 4.0000% | 3.0000% | 3.0000% |
| Octenylsuccinate | | | | | | |
| Aristoflex AVC ³ | 0.7500% | | 0.7500% | 0.7500% | 0.7500% | 0.7500% |
| Cyclomethicone | | | | | | |
| Dow Corning 1503 ⁴ | 2.0000% | 2.0000% | 2.0000% | 2.0000% | 2.0000% | 2.0000% |
| FD&C Blue No. 1 (CI | 0.0002% | 0.0002% | 0.0002% | 0.0002% | 0.0002% | 0.0002% |
| 42090) | | | | | | |
| Mackstat DM ⁵ | 0.0800% | 0.0800% | 0.0800% | 0.0800% | 0.0800% | 0.0800% |
| Glydant Plus Liquid ⁶ | 0.3200% | 0.3200% | 0.3200% | 0.3200% | 0.3200% | 0.3200% |
| KTZ Interfine Gold ⁷ | 0.1000% | 0.1000% | 0.1000% | 0.1000% | 0.1000% | 0.1000% |
| KTZ Interfine Green ⁸ | 0.5000% | 0.5000% | 0.5000% | 0.5000% | 0.2500% | 0.2500% |
| 1-Phenylimidazole | 0.5000% | 1.5000% | | 1.00 | 0.7500% | 0.2500% |
| 4-Phenylimidazole | 0.5000% | | 1.5000% | 0.7500% | 0.7500% | 0.7500% |
| Phase D | | | | | | |
| Fragrance | 0.8000% | 0.8000% | 0.8000% | 0.8000% | 0.8000% | 0.8000% |
| Menthol | 0.0500% | 0.0900% | 0.0900% | 0.0900% | 0.0500% | 0.0900% |
| Menthyl Lactate | 0.1500% | 0.2000% | 0.2000% | 0.2000% | 0.2500% | 0.2000% |
| | Sample | Sample | Sample | Sample | Sample | Sample |
| | 7 | 8 | 9 | 10 | 11 | 12 |
| Phase A | | | | | | |
| Water | Qs | Qs | Qs | Qs | Qs | Qs |
| Glycerin | 4.0000% | 4.0000% | 2.0000% | 2.0000% | 3.0000% | 2.0000% |
| Sorbitol | | | 2.0000% | 2.0000% | 2.0000% | 2.0000% |
| Disodium EDTA | 0.1000% | 0.1000% | 0.0500% | 0.0500% | 0.0500% | 0.0500% |
| Phase B | | | | | | |
| Cetearyl Alcohol | | | 1.0000% | 1.0000% | 1.0000% | 1.0000% |
| Emulgade Pl 68/50 ¹ | 0.2000% | 0.2000% | | | | |
| Cetiol SN ² | 5.0000% | 5.0000% | 1.5000% | 1.5000% | 1.5000% | 3.5000% |
| Cetyl Alcohol | 0.8900% | 0.8900% | | | | |
| PEG-100 Stearate | 0.1000% | | | | | |
| Polymethylsilsesquioxane | 1.0000% | 1.0000% | | | 1.0000% | 1.0000% |
| Sorbitan Stearate | 0.1000% | 0.1000% | | | | |
| Steareth-2 | | | 1.8000% | 1.8000% | 1.8000% | 1.8000% |
| Steareth-21 | | | 0.9000% | 0.9000% | 0.9000% | 0.9000% |
| Stearic acid | 0.1000% | | | | | |
| Stearyl Alcohol | 0.6100% | 0.6100% | | | | |
| Phase C | | | | | | |
| Aluminum Starch | 3.0000% | 3.0000% | | | | |
| Octenylsuccinate | | | | | | |
| Aristoflex AVC ³ | 0.7500% | | 0.7500% | | | |
| Cyclomethicone | | | 2.0000% | 2.0000% | | |
| Dow Corning 1503 ⁴ | 2.0000% | 2.0000% | 0.5000% | 0.5000% | 0.5000% | 0.5000% |
| FD&C Blue No. 1 (CI | 0.0002% | 0.0002% | 0.0002% | 0.0002% | 0.0002% | 0.0002% |
| 42090) | | | | | | |
| Mackstat DM ⁵ | 0.0800% | 0.0800% | 0.0800% | 0.0800% | 0.0800% | 0.0800% |
| Glydant Plus Liquid ⁶ | 0.3200% | 0.3200% | 0.3200% | 0.3200% | 0.3200% | 0.3200% |
| KTZ Interfine Gold ⁷ | 0.1000% | 0.1000% | | | 0.1000% | |
| KTZ Interfine Green ⁸ | 0.5000% | 0.5000% | | | 0.2500% | |
| 1-Phenylimidazole | 0.5000% | 1.5000% | | 1.0000% | 0.7500% | 0.2500% |
| 4-Phenylimidazole | 0.5000% | | 1.5000% | 0.7500% | 0.7500% | 0.7500% |
| Phase D | | | | | | |
| Fragrance | 0.8000% | 0.8000% | 0.4000% | 0.4000% | 0.4000% | 0.4000% |
| Menthol | 0.0900% | 0.0500% | | | 0.0500% | 0.0500% |
| Menthyl Lactate | 0.2000% | 0.1500% | | | 0.1500% | 0.1500% |

¹ Cetearyl Glucoside (and) Cetearyl Alcohol from Cognis Corp, Cincinnati, OH
² Cetearyl Isononanoate from Cognis Corp, Cincinnati, OH
³ Ammonium Acryloyldimethyltaurate/VP Copolymer from Clariant International AG, Switzerland.
⁴ Dimethicone (and) Dimethiconol from Dow Corning, Midland, MI
⁵ DMDM Hydantoin (and) Water from Rhodia Inc, Cranbury. NJ
⁶ DMDM Hydantoin (and) Iodopropynyl Butylcarbamate (and) Water from Lonza Group Ltd, Switzerland
⁷ Mica (and) Titanium dioxide (and) Tin Oxide from Kobo Products, Plainfield, NJ
⁸ Mica (and) Titanium dioxide from Kobo Products, Plainfield, NJ

### Example Set B

### Washing Compositions

TABLE 1B
Washing Composition Ingredients

| | | Sample | Sample | Sample | Sample |
|---|---|---|---|---|---|
| Ingredient | Sample 1 | 2 | 3 | 4 | 5 |
| Water | Qs | Qs | Qs | Qs | Qs |
| Polyquaternium-10 | 0.25 | 0.25 | 0.25 | 0.25 | 0.25 |
| (JR-400) | | | | | |
| Disodium EDTA | 0.10 | 0.10 | 0.10 | 0.10 | 0.10 |
| PEG-100 | 0.20 | 0.20 | 0.20 | 0.20 | 0.20 |
| Sorbitol¹³ | 1.99 | 1.99 | 1.99 | 1.99 | 1.99 |
| Glycerin | 1.99 | 1.99 | 1.99 | 1.99 | 1.99 |
| Sodium | 3.19 | 3.19 | 3.19 | 3.19 | 3.19 |
| Lauroamphoacetate¹⁴ | | | | | |
| Sodium Trideceth | 3.24 | 3.24 | 3.24 | 3.24 | 3.24 |
| Sulfate¹⁵ | | | | | |
| Sodium Myristol | 1.49 | 1.49 | 1.49 | 1.49 | 1.49 |
| Sarcosinate¹⁶ | | | | | |
| Lauric Acid | 0.15 | 0.15 | 0.15 | 0.15 | 0.15 |
| Citric Acid | 0.50 | 0.50 | 0.50 | 0.50 | 0.50 |
| PEG-200 | 2.99 | 2.99 | 2.99 | 2.99 | 2.99 |
| Hydrogenated | | | | | |
| Glyceryl Palmitate/ | | | | | |
| PEG-7 Glyceryl | | | | | |
| Cocoate¹⁷ | | | | | |
| DMDM Hydantoin + | 0.40 | 0.40 | 0.40 | 0.40 | 0.40 |
| Iodopropynyl | | | | | |
| Butalcarbamate | | | | | |
| Perfume | 0.50 | 0.50 | 0.50 | 0.50 | 0.50 |
| 1-Phenylimidazole | 0.50 | 1.50 | 0.00 | 1.00 | 0.75 |
| 4-Phenylimidazole | 0.50 | 0.00 | 1.50 | 0.75 | 0.75 |
| Menthol | 0.00 | 0.10 | 0.10 | 0.08 | 0.04 |

¹³Sorbitol 70% Solution
¹⁴Sodium Lauroamphoacetate 32% Solution
¹⁵Sodium Trideceth Sulfate 65% Solution
¹⁶Sodium Myristol Sarcosinate 30% Solution
¹⁷Antil 200 - (Evonik/Goldschmidt)

Making instructions for Washing Composition

Weigh out the water in a vessel sufficient to hold the entire batch. Insert an overhead mixer with impeller into the vessel and increase agitation to create a vortex. Sprinkle the polymer into the vortex, ensure well dissolved. Heat batch to about 60° C. to hydrate the polymer. Add EDTA, PEGs, Sorbitol, Glycerin, Sodium Lauroamphoacetate, and the surfactants while heating. After batch is at 60° C., add the lauric acid. Continue mixing at 60° C. for at least five minutes. Adjust to a pH from 5.9-6.5 with citric acid and/or water. Remove heat, allow to cool to 35° C. Once below 35° C., add the perfume, preservatives and other ingredients.

### Example Set C

### Pre-Shave Prep Examples

TABLE 1C
Pre-Shave Prep Ingredients

| | | | Sample | Sample | Sample |
|---|---|---|---|---|---|
| Ingredient | Sample 1 | Sample 2 | 3 | 4 | 5 |
| Water | Qs | Qs | Qs | Qs | Qs |
| Sepigel 305 | 2.00 | 2.00 | 2.00 | 2.00 | 2.00 |
| (Polyacrylamide | | | | | |
| & C13-14 | | | | | |
| Isoparaffin & | | | | | |
| Laureth-7) | | | | | |
| Polyox N12K | 0.50 | 0.50 | 0.50 | 0.50 | 0.50 |
| (PEG-23M) | | | | | |
| Natrosol 250 | 0.80 | 0.80 | 0.80 | 0.80 | 0.80 |
| HHR (HEC) | | | | | |
| Glycerin 99.7% | 5.00 | 5.00 | 5.00 | 5.00 | 5.00 |
| Usp/Fcc | | | | | |
| Brij 35 (Laureth- | 2.00 | 2.00 | 2.00 | 2.00 | 2.00 |
| 23) | | | | | |
| Disodium EDTA | 0.10 | 0.10 | 0.10 | 0.10 | 0.10 |
| Perfume | 0.15 | 0.15 | 0.15 | 0.15 | 0.15 |
| Glydant Plus | 0.20 | 0.20 | 0.20 | 0.20 | 0.20 |
| 1-Phenylimidazole | 0.50 | 1.50 | 0.00 | 1.00 | 0.75 |
| 4-Phenylimidazole | 0.50 | 0.00 | 1.50 | 0.75 | 0.75 |
| Menthol | 0.00 | 0.05 | 0.05 | 0.04 | 0.02 |

The pre-shave prep samples above are made according to the method below.

Weigh out the water in a vessel sufficient to hold the entire batch. Insert an overhead mixer with impeller into the vessel and increase agitation to create a vortex. Pre-blend the thickener and polymer powders. Sprinkle the polymer blend into the vortex until incorporated. Begin heating batch to 70 C to hydrate the polymers. Once the batch is at 70 C, add the oil and mix until uniform and dispersed. Add the liquid dispersion polymer to the batch and mix until uniform and hydrated, increasing rpms to maintain good mixing. Add the surfactant and mix until uniform and dispersed. Begin cooling batch to below 45 C. Once below 45 C, add the perfume, preservatives and other temperature-sensitive additives. Cool to below 35 C and QS with water.

### Example Set D

### Post Foaming Shave Gel Examples

Making instructions can be found in US 2006/0257349, paragraph 21.

TABLE 1D
Post Foaming Shave Gel Ingredients

| Ingredient | Sample 1 | Sample 2 | Sample 3 | Sample 4 | Sample 5 |
|---|---|---|---|---|---|
| Sorbitol 70% Solution | 0.97% | 0.97% | 0.97% | 0.97% | 0.97% |
| Glycerin | 0.49% | — | 0.49% | — | 0.49% |
| Water | QS | QS | QS | QS | QS |
| hydroxyethyl cellulose¹⁸ | 0.49% | 0.49% | 0.49% | 0.49% | 0.49% |
| PEG-90M¹⁹ | 0.06% | 0.06% | 0.06% | 0.06% | 0.06% |
| PEG-23M²⁰ | 0.05% | 0.05% | 0.05% | 0.05% | 0.05% |
| PTFE²¹ | 0.15% | 0.15% | 0.15% | 0.15% | 0.15% |
| Palmitic acid | 7.53% | 7.53% | 7.53% | 7.53% | 7.53% |
| Stearic Acid | 2.53% | 2.53% | 2.53% | 2.53% | 2.53% |
| Glyceryl Oleate | 1.94% | 1.94% | 1.94% | 1.94% | 1.94% |
| Triethanolamine (99%) | 5.88% | 5.88% | 5.88% | 5.88% | 5.88% |
| Lubrajel Oil²² | 0.49% | 0.97% | 0.49% | 0.97% | 0.49% |
| 1-Phenylimidazole | 0.50% | 1.50% | 0.00% | 1.00% | 0.75% |
| 4-Phenylimidazole | 0.50% | 0.00% | 1.50% | 0.75% | 0.75% |
| Menthol | — | 0.11% | — | 0.11% | — |
| Fragrance | 0.87% | 0.87% | 0.87% | 0.87% | 0.87% |
| Other (e.g. Vit E, Aloe, | 0.10% | 0.10% | 0.10% | 0.10% | 0.10% |
| etc.) | | | | | |
| Dye | 0.10% | 0.10% | 0.10% | 0.10% | 0.10% |
| Isopentane (and) | 2.8500% | 2.8500% | 2.8500% | 2.8500% | 2.8500% |
| Isobutane | | | | | |

¹⁸Available as Natrosol 250 HHR from Hercules Inc., Wilmington, DE
¹⁹Available as Polyox WSR-301 from Amerchol Corp., Piscataway, NJ
²⁰Available as Polyox WSR N-12K from Amerchol Corp., Piscataway, NJ
²¹Available as Microslip 519 from Micro Powders Inc., Tarrytown, NY
²²Available from Guardian Laboratories, Hauppauge, NY

### Example 2

### In Vitro CYP Inhibition Assay

### Materials & Methods

Microsomal preparation: One lobe of fresh pig liver was obtained at the time of slaughter from a food-processing company and immediately placed in ice cold 15 mM KH2PO4/250 mM sucrose (pH 7.4) and kept on ice during transportation. A 10 g sample of liver was minced and homogenized in 30 mls of homogenization buffer (15 mM KH2PO4/250 mM sucrose) using a Tekmar homoginizer by pulsing 3 times 20 second pulses. This procedure was repeated for a total of 8×10 g samples of pig liver. The remaining pig liver was cut into 10-g pieces and wrapped in aluminum foil and stored at −80° C. The homogenates from the 8 samples were pooled and centrifuged at 13,000×g for 20 minutes at 4° C. to remove crude debris and the supernatant was further centrifuged at 100,000×g for 70 minutes at 4° C. The microsomal pellets were resuspended into 50 mls of 150 mM KH2PO4/1 mM DTT (pH 7.4) and 1 mL aliquots were stored at −80° C.
In vitro 4-hydroxylase activity assay: All procedures were carried out under minimal light in order to prevent degradation of the retinoid samples. The ATRA 4-hydroxylase assay can be described briefly as follows: 100-150 μg of pig liver microsomal protein in 150 mM KH2PO4 was incubated at 37° C. in the presence of radiolabeled ATRA and 5 mM NADPH for 90 min. The final ATRA concentration was 1 μM, as a combination of [20-Methyl-³H]ATRA and unlabeled ATRA. Initially radiolabeled ATRA was used to assist in validating the method. Once retention times of retinoid metabolites were identified unlabeled ATRA was used for screening. Compounds tested as possible competitive substrates were added to the assay 10 min prior to the addition of ATRA. Ethanol, containing 0.1% butylated hydroxytoluene (BHT) as an antioxidant, was used to stop the reactions. For recovery of ATRA the pH of reactions were adjusted to 3.0 before extraction with 0.1N HCl. Retinoids were extracted from the protein aqueous phase with 4 mls hexane, 1 ml 100 μg/ml BHT in water (pH 3). An additional 4 mls of hexane (pH 3) was used for a second extraction. The organic extractions were pooled, evaporated to dryness using a speed vac, and resuspended in 62.5 μl acetonitrile containing 1 mg/ml BHT. Samples were analyzed by HPLC.
HPLC separation of retinoids: An HPLC system (Waters Corp., Milford Mass.) containing a Vydac 201 TP54 column (15 cm×46 mm), mutiwavelength detector (Waters 490) set at 350 nm, and a β-RAM detector (IN/US Systems, Tampa, Fla.) was used to separate retinoids. Mobile phases used for gradient elution of retinoids were those of Duell et al., J. Clin. Invest. 1992 October; 90(4): 1269-1274. Mobile phase A was acetonitrile:0.02 M ammonium acetate:acetic acid (1:1:0.01) and mobile phase B was acetonitrile:0.2M ammonium acetate;acetic acid (19:1:0.008). At the start of run solvent A was run 100% followed by a linear gradient to solvent B at 3 min, a shallow gradient to 81% solvent B at 38 min, and 100% solvent B at 40 min. The flow rate was 0.5 ml/min and the total time for separation was 60 min. Effluent from the HPLC column flowed directly into a flow-through scintillation spectrometer (β-RAM). Because of the sensitivity limitations of the β-RAM spectrometer, it was not used to quantitate peak areas but rather to confirm ATRA and ATRA 4-hydroxylase activity. Calculated peaks areas (using Millenium software—Waters Corp.) of ATRA and ATRA metabolites were used to quantify relative activity levels.

As presented in Table 2, several compounds were screened through the in vitro hydroxylase assay. Ketoconazole was used as a positive control, while climbazole is a known 4-hydroxylase inhibitor marketed as an antifungal active.

TABLE 2
Screening results from in vitro 4-hydroxylase inhibition assay

| Compound | IC₅₀ (μM) |
|---|---|
| Ketoconazole | 8.8 |
| Climbazole | 47.5 |
| 1-phenylimidazole | 52.5 |
| 4-phenylimidazole | 58.8 |
| 1-benzylimidazole | 77.5 |
| 4,5-diphenylimidazole | 100.0 |
| 1-benzyl-2-methylimidazole | 307.5 |
| Clotrimazole | >500 |
| 2-phenylimidazole | >500 |

### Example 3

### In Vitro CYP/CYP3A4 Inhibition Assay

The commercially available P450-GLO™ Assay kit (Promega Corporation, Madison Wis.) was used to screen various compounds for potential CYP activity, specifically CYP3A4A inhibition activity. CYP3A4A is thought to be one of the primary CYP isoforms responsible for retinoic acid metabolism in the skin. Three benchmark agents, liarozole, climbazole, and ketoconazole, were assessed for CYP3A4 inhibition to confirm that the inhibition activity (the IC₅₀ for CYP3A4 inhibition) measured by the assay corresponds to the activity reported by the published literature.

The results set forth in Table 3 show that the substituted azole compounds having the specific structure set forth herein are CYP inhibitors, and thus function as RAMBAs.

TABLE 3
Screening results from in vitro CYP/CYP3A4 Inhibition Assay

| | | | IC₅₀ |
|---|---|---|---|
| Structure | Compound | CAS No. | ( uM) |
| 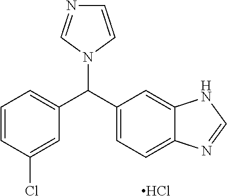 [SMILES: Clc1cccc(C(c2ccc3nc[nH]c3c2)n2ccnc2)c1 ; Cl] | Liarizole hydrochloride | 145858-50-0 | Hit <0.1 uM (0.08- 0.1 uM) |
| 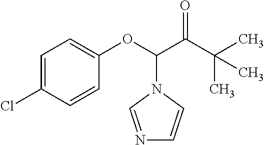 [SMILES: CC(C)(C)C(=O)C(Oc1ccc(Cl)cc1)n1ccnc1] | Climbazole | 38083-17-9 | Hit 0.1 uM |
| 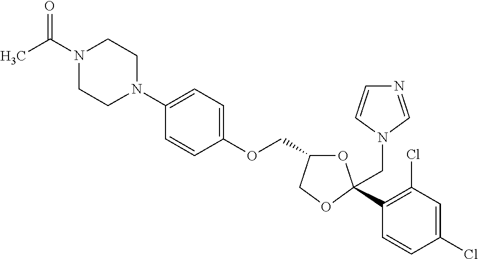 [SMILES: CC(=O)N1CCN(c2ccc(OC[C@H]3CO[C@](Cn4ccnc4)(c4ccc(Cl)cc4Cl)O3)cc2)CC1] | Ketoconozole | 65277-42-1 | Hit 0.5 uM |
| 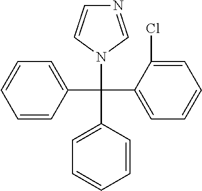 [SMILES: Clc1ccccc1C(c1ccccc1)(c1ccccc1)n1ccnc1] | Clotrimazole | 23593-75-1 | Hit 0.3 uM |
| 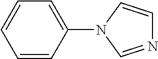 [SMILES: c1ccc(-n2ccnc2)cc1] | 1-Phenylimidazole | 7164-98-9 | Hit l uM |
| 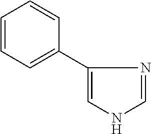 [SMILES: c1ccc(-c2c[nH]cn2)cc1] | 4-Phenylimidazole | 670-95-1 | Hit 1.5 uM |
| 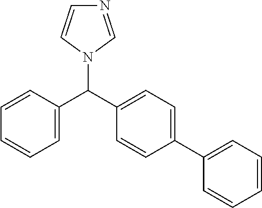 [SMILES: c1ccc(-c2ccc(C(c3ccccc3)n3ccnc3)cc2)cc1] | Bifonazole | 60628-96-8 | Hit 0.8 uM |
| 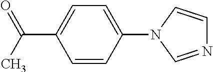 | 4′-(Imidazol-1yl) acetophenome | 10041-06-2 | Hit 0.8 uM |
| 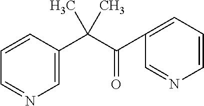 [SMILES: CC(C)(C(=O)c1cccnc1)c1cccnc1] | Metyrapone | 54-36-4 | Hit 3 uM |
| 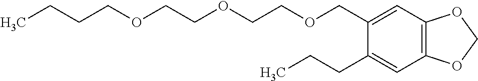 [SMILES: CCCCOCCOCCOCc1cc2c(cc1CCC)OCO2] | Piperonyl butoxide | 51-03-6 | Hit 3 uM |
| 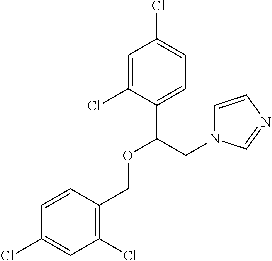 [SMILES: Clc1ccc(COC(Cn2ccnc2)c2ccc(Cl)cc2Cl)c(Cl)c1] | Miconazole | 22916-47-8 | Hit 5 uM |
| 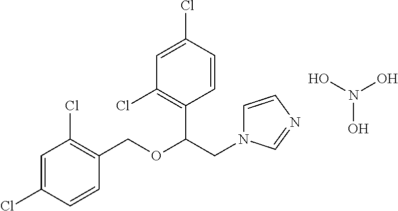 | Miconazole Nitrate | 75319-48-1 | Hit 5 uM |
| 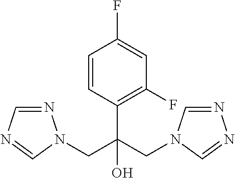 [SMILES: OC(Cn1cnnc1)(Cn1cncn1)c1ccc(F)cc1F] | Fluconazole | 86386-73-4 | Hit ~10 uM |
| 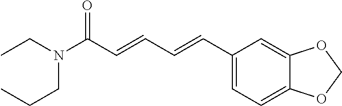 [SMILES: CCCN(CC)C(=O)/C=C/C=C/c1ccc2c(c1)OCO2] | Piperine | 94-62-2 | Hit ~10 uM |
| 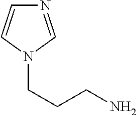 | N-(3-Aminopropyl) imidazole | 5036-48-6 | no hit >10 uM |
| 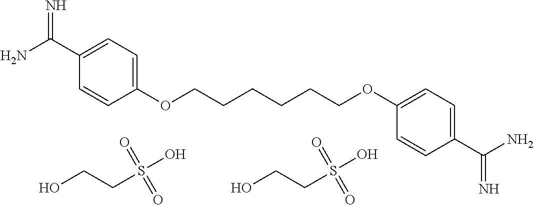 | Hexamidine diisethionate | 659-40-5 | no hit >10 uM |
| 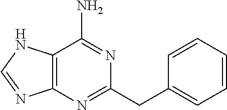 [SMILES: Nc1nc(Cc2ccccc2)nc2nc[nH]c12] | 3-Benzyladenine | 7280-81-1 | no hit >10 uM |
| 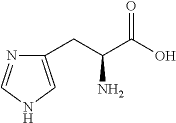 [SMILES: N[C@@H](Cc1c[nH]cn1)C(=O)O] | Histidine | 71-00-1 | no hit >10 uM |
| 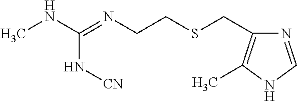 [SMILES: CN/C(=N/CCSCc1nc[nH]c1C)NC#N] | Cimetidine | 51481-61-9 | Near Hit IC50 > 10 uM |
| 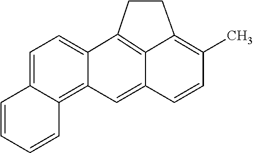 [SMILES: Cc1ccc2cc3c(ccc4ccccc43)c3c2c1CC3] | Methylcholanthrene | 56-49-5 | Near Hit > 10 uM |
| 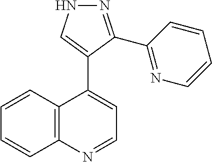 [SMILES: c1ccc(-c2n[nH]cc2-c2ccnc3ccccc23)nc1] | LY-364947 | 396129-53-6 | no hit >10 uM |
| 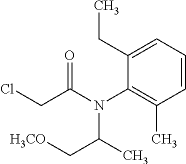 [SMILES: CCc1cccc(C)c1N(C(=O)CCl)C(C)COC] | Metolachlor | 52118-45-2 | no hit >10 uM |
| 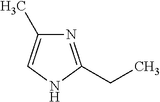 | 2-Ethyl-4- methylimidazole | 931-36-2 | no hit >10 uM |
| 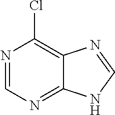 | 6-Chloropurine | 87-42-3 | no hit >10 uM |
| 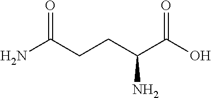 | L-Glutamine | 56-85-9 | no hit >10 uM |
| 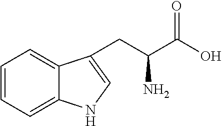 [SMILES: N[C@@H](Cc1c[nH]c2ccccc12)C(=O)O] | L-Tryptophan | 73-22-3 | no hit >10 uM |
| 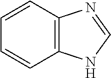 | Benzimidazole | 51-17-2 | no hit >10 uM |
| 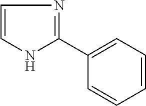 [SMILES: c1ccc(-c2ncc[nH]2)cc1] | 2-Phenylimidazole | 670-96-2 | no hit >10 uM |
| 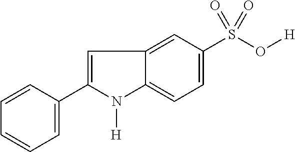 [SMILES: O=S(=O)(O)c1ccc2[nH]c(-c3ccccc3)cc2c1] | 2-Phenyl benzimidazole | 716-79-0 | no hit >10 uM |
| 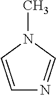 | 1-Methylimidazole | 616-47-7 | no hit >10 uM |
| 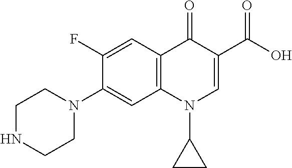 [SMILES: O=C(O)c1cn(C2CC2)c2cc(N3CCNCC3)c(F)cc2c1=O] | Ciprofloxacin HCl | 85721-33-1 | no hit >10 uM |
| 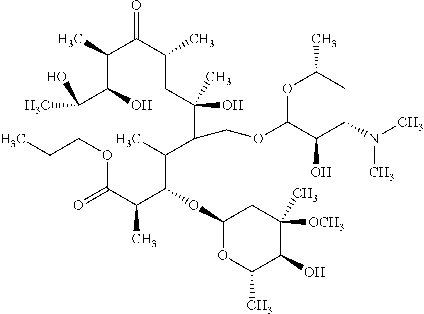 | Erythromycin | 114-07-8 | no hit >10 uM |
| 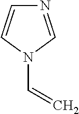 | 1-Vinylimidazole | 1072-63-5 | no hit >10 uM |
| 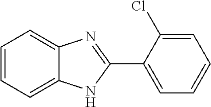 [SMILES: Clc1ccccc1-c1nc2ccccc2[nH]1] | 2-(2-Chlorophenyl) benzimidazole | 3674-96-7 | no hit >10 uM |
| 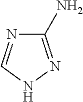 | Amitrol | 61-82-5 | no hit >10 uM |
| 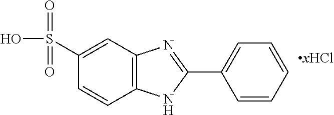 [SMILES: O=S(=O)(O)c1ccc2[nH]c(-c3ccccc3)nc2c1 ; "·xHCl"] | 2-Pheny1-5- benzimidazole sulfonic acid | 27503-81-7 | no hit >10 uM |
| 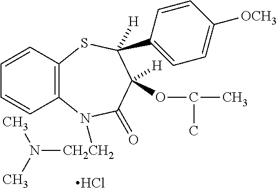 | Diltiazem | 42399-41-7 | no hit >10 uM |
| 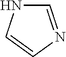 | Imidazole | 288-32-4 | no hit >10 uM |
| 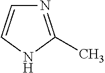 | 2-Methylimidazole | 693-98-1 | no hit >10 uM |
| 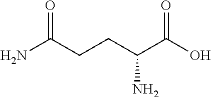 | D-Glutamine | 5959-95-5 | no hit >10 uM |
| 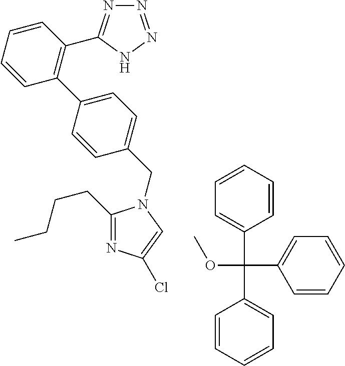 | 2-Butyl-4-chloro-5- (hydroxymethyl) imidazole | 79047-41-9 | no hit >10 uM |
| 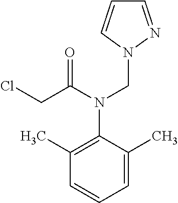 [SMILES: Cc1cccc(C)c1N(Cn1cccn1)C(=O)CCl] | Metazachlor | 67129-08-2 | no hit >10UM |
| 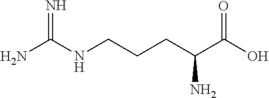 [SMILES: N=C(N)NCCC[C@H](N)C(=O)O] | L-Arginine | 74-79-3 | no hit >10 uM |
| 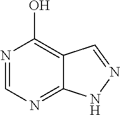 | Allopurinol | 315-30-0 | no hit >10 uM |
| 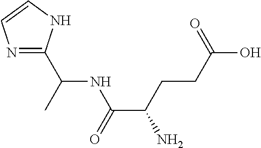 [SMILES: CC(NC(=O)[C@@H](N)CCC(=O)O)c1ncc[nH]1] | Glutamylamidoethyl imidazole | 169283-81-2 | no hit >10 uM |
| 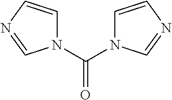 [SMILES: O=C(n1ccnc1)n1ccnc1] | 1′-1- Carbonylimidazole | 530-62-1 | no hit >10 uM |
| 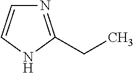 | 2-Ethylimidazole | 1072-62-4 | no hit >10 uM |
| 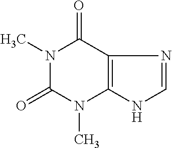 | Theophylline | 58-55-9 | no hit >10 uM |
| 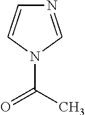 | 1-Acetylimidazole | 2466-76-4 | no hit >10 uM |
| 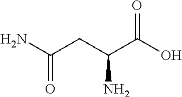 | L-Asparigine | 70-47-3 | no hit >10 uM |
| 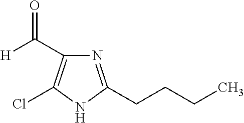 | 2-Butyl-4-chloro-5- formylimidazole | 83857-96-9 | no hit >10 uM |
| 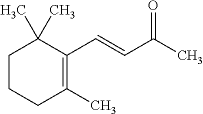 | β-Ionone | 79-77-6 | no hit >10 uM |
| 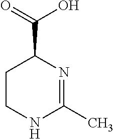 | Ectoine | 96702-03-3 | no hit >10 uM |
| 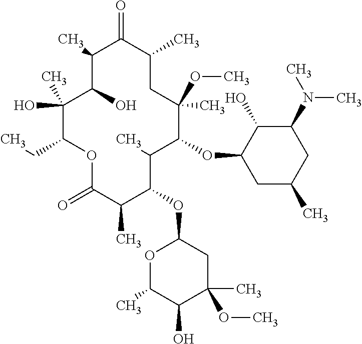 [SMILES: CC[C@H]1OC(=O)[C@H](C)[C@@H](O[C@H]2C[C@@](C)(OC)[C@@H](O)[C@H](C)O2)C(C)[C@@H](O[C@@H]2C[C@H](C)C[C@H](N(C)C)[C@H]2O)[C@](C)(OC)C[C@@H](C)C(=O)[C@H](C)[C@@H](O)[C@]1(C)O] | Clarithromycin | 81103-11-9 | no hit >10 uM |
| 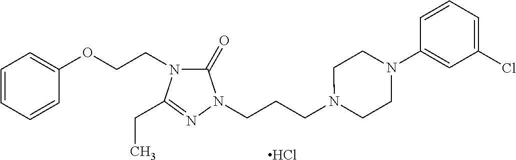 [SMILES: CCc1nn(CCCN2CCN(c3cccc(Cl)c3)CC2)c(=O)n1CCOc1ccccc1 ; Cl] | Nefazodone HC1 | 83366-9 | no hit >10 uM |
| 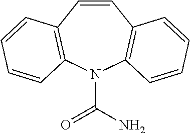 [SMILES: NC(=O)N1c2ccccc2C=Cc2ccccc21] | Carbamazepine | 298-46-4 | no hit >10 uM |
| 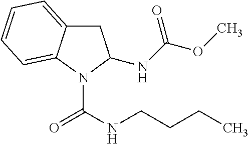 [SMILES: CCCCNC(=O)N1c2ccccc2CC1NC(=O)OC] | Benomyl | 17804-35-2 | no hit >10 uM |
| 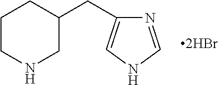 [SMILES: c1nc(CC2CCCNC2)c[nH]1 ; "·2HBr"] | Immepip dihydrobromide | 164391-47-3 | no hit >10 uM |
| 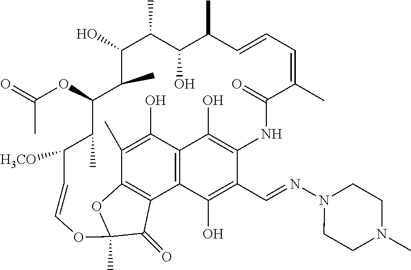 [SMILES: CO[C@H]1/C=C/O[C@@]2(C)Oc3c(C)c(O)c4c(O)c(c(/C=N/N5CCN(C)CC5)c(O)c4c3C2=O)NC(=O)/C(C)=C\C=C\[C@H](C)[C@H](O)[C@@H](C)[C@@H](O)[C@@H](C)[C@H](OC(C)=O)[C@@H]1C] | Rifampicin | 13292-46-1 | no hit >10 uM |
| 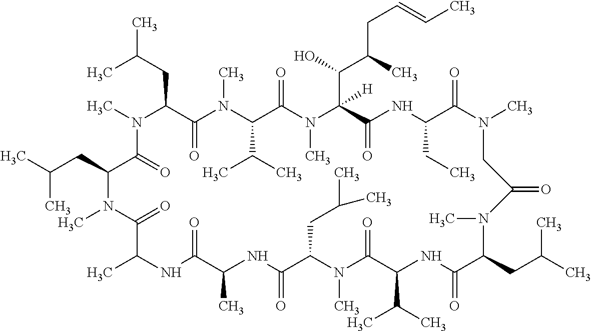 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)NC(C)C(=O)N(C)[C@@H](CC(C)C)C(=O)N(C)[C@@H](CC(C)C)C(=O)N(C)[C@@H](C(C)C)C(=O)N1C] | Cyclosporin A | 59865-13-3 | no hit >10 uM |
| 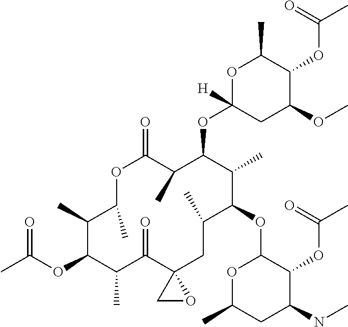 [SMILES: CN[C@H]1C[C@@H](C)OC(O[C@@H]2[C@@H](C)[C@H](O[C@H]3C[C@H](OC)[C@@H](OC(C)=O)[C@H](C)O3)[C@@H](C)C(=O)O[C@H](C)[C@H](C)[C@H](OC(C)=O)[C@@H](C)C(=O)[C@]3(CO3)C[C@@H]2C)[C@@H]1OC(C)=O] | Troleandomycin | 2751-09-9 | no hit >10 uM |
| 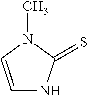 | Methimazole | 60-56-0 | no hit >10 uM |
| 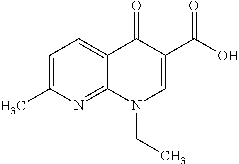 [SMILES: CCn1cc(C(=O)O)c(=O)c2ccc(C)nc21] | Nalidixic acid | 389-08-2 | no hit >10 uM |
| 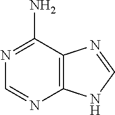 | Adenine | 73-24-5 | no hit >10 uM |
| 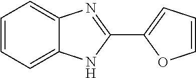 [SMILES: c1coc(-c2nc3ccccc3[nH]2)c1] | Fuberidazole | 3878-19-1 | no hit >10 uM |
| 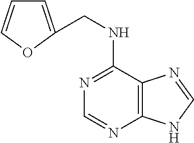 [SMILES: c1coc(CNc2ncnc3[nH]cnc23)c1] | Kinetin | 525-79-1 | no hit >10 uM |

### Example 4

### Method of Treatment

A test subject topically applies a shaving cream composition comprising 0.55% 1-phenylimidazole, by weight in a vehicle, to the facial and neck regions prior to shaving. A leave-on skin cream comprising 0.55% 1-phenylimidazole, by weight in a vehicle, is applied to the same facial and neck regions after shaving. After 8 weeks of treatment, the subject notes that his facial and neck skin are significantly less inflamed.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. In particular, U.S. Provisional Application Ser. No. 61/762,562 is incorporated herein by reference in its entirety. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of alleviating mechanically-induced skin inflammation comprising:
a. identifying a target skin surface subject to mechanically-induced inflammation;
b. applying to said skin surface a composition comprising:
i. an effective amount of a substituted azole compound represented by the structure: 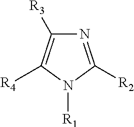
where:
R1 is an alkyl or phenyl electron donating group
R2 is hydrogen
R3 and R4 do not form a fused ring, and each is independently selected from the group consisting of H, alkyl, and phenyl; and
ii. a dermatologically acceptable carrier;
wherein said composition is applied for a period of time sufficient to improve the appearance of one or more signs of mechanically-induced inflammation.

2. The method of claim 1, wherein said substituted azole compound is 1-phenylimidazole, 4-phenylimidazole, or a combination thereof.

3. The method of claim 2, wherein the skin subject to mechanically-induced inflammation comprises shaved skin.

4. The method of claim 3, wherein said shaved skin comprises facial skin or neck skin.

5. The method of claim 3, wherein said composition additionally comprises a sunscreen active.

6. The method of claim 3, wherein said composition additionally comprises an optional anti-inflammatory agent.

7. The method of claim 3, wherein said composition further comprises a skin tone agent.

8. The method of claim 7, wherein said skin tone agent is selected from the group consisting of vitamin B3 compounds, sugar amines, hexamidine compounds, salicylic acid, 1,3-dihydroxy-4-alkylbenzene, xanthenes, and combinations thereof.

9. The method of claim 3, additionally comprising a step of applying a second composition to the skin surface.

10. The method of claim 9, wherein the second composition comprises a sunscreen active, an optional anti-inflammatory agent, a skin tone agent, or a combination thereof.
